(19) United States
(12) Patent Application Publication
(10) Pub. No.: US 2023/0143130 A1
(43) Pub. Date: May 11, 2023

(54) SYSTEM AND METHOD FOR IDENTIFYING FRUIT SHELF LIFE

(71) Applicant: AGROPEEPER TECHNOLOGIES PRIVATE LIMITED, Navi Mumbai, Maharashtra (IN)

(72) Inventors: Varsha BHOLE, Navi Mumbai (IN); Arun KUMAR, Udaipur (IN)

(73) Assignee: AGROPEEPER TECHNOLOGIES PRIVATE LIMITED, Navi Mumbai, Maharashtra (IN)

(21) Appl. No.: 17/917,999

(22) PCT Filed: Mar. 23, 2021
(86) PCT No.: PCT/IN2021/050302

§ 371 Date: Oct. 10, 2022

(30) Foreign Application Priority Data
Apr. 10, 2020 (IN) 202021015721

(51) Int. Cl. G01N 33/02; G01N 21/3563; G01N 25/20

(52) U.S. Cl. CPC G01N 33/025; G01N 21/3563; G01N 25/20

(57) ABSTRACT

The present invention relates to a method for identifying fruit shelf life automatically based on thermal imaging Identifying every day the pattern of change in temperature of fruit's thermal image, we can predict the shelf life of fruit which is how many days the fruit will remain edible in an accurate manner without destructing the fruit. In this system, thermal dataset is created comprising of samples of thermal images (206) of fruit taken on every day after harvesting where fruit may be from cold storage or room temperature using thermal imaging device (204). Transfer learning a deep learning technique is applied on this thermal dataset to compute the threshold weights (210) which are then used to classify or predict the fruit shelf life by comparing these pre-trained weights with the features of fruit's thermal image extracted from convolution and poling layer (212) of deep learning model

### TECHNICAL FIELD

The present invention generally relates to fruits shelf life and in particularly, the present invention relates to method and system for determining fruit shelf life using thermal imaging, deep learning techniques.

### BACKGROUND ART

In today's consumer lifestyle, food habits have become an important part to dictate health of the individuals. Natural food like fruits and vegetables supplement most of the need for this healthy lifestyle; however the consumer does not often have access to the right quality of fruits and/or vegetables. Apart from increasing the shelf life, identifying the right time to harvest fruits depending on their maturity helps fruit marketability with better taste levels of the fruit. Current methods of grading fruits based on maturity are mostly by visual inspection of physical characteristics like colour, size and shape and using traditional image processing methods for classification.

Agricultural products are graded based on their dimensions and quality. This grade is used to sort them and assign them to different sales channels. Each item may yield better income based when properly allocated according to its exact characteristics. Usually, higher grade and bigger agricultural products generate larger revenues. Traditional grading was human-dependent. Later, mechanical devices were used to differentiate agricultural products based on their dimensions and weight. Such devices are still in use today as a reliable method for grading and sorting. More recently, as image processing algorithms emerged, visual inspection techniques were added to perfect the process. They often were manually-tuned and provided a substitute to the human eye, enabling to detect many defects, which humans cannot detect when pace becomes faster. The new wave of intelligent algorithms for grading and sorting is much more powerful than traditional visual analysis algorithms: they have automatic learning capabilities, which ensure a detection performance far beyond the speed and accuracy of any trained operator.

Many analytical models have been technologically advanced for such a task using machine learning techniques where RGB imaging has the only basis. The existing system is slow based on Machine Learning.

In one solution, a method is disclosed for analyzing multiple significant maturity indices like color, ultra-structural changes in texture of the fruit to measure precisely the shelf life, optimum harvest time and develop a framework based on deep learning techniques for feature extraction and classification of fruits. Low cost standalone farm management solutions can be built using this concept that will help multiple stakeholders in the fruit supply chain in improved quality control, grading and classification of fruits.

In the prior work, the shelf life has been determined by extracting the color features of the fruit and then classified it into appropriate classes. But it has been observed that for the same category of fruit, there has been a huge difference in its surface color of same class of fruits which might be giving the less accurate results. Also, we can take an example of Langra mango where in its all shelf-lives skin color remains green. So it is difficult to classify the shelf-life of fruit using an only external surface color which is a non-destructive technique. Langra mango retains its green color after it gets ripe, so it is difficult to find a shelf-life using external skin color. Therefore, there exists a need to have a better system and method for identifying fruit shelf life.

### SUMMARY OF INVENTION

The present invention generally relates to an image processing with the help of deep learning classification technique. In particularly, the present invention relates to a system for identifying fruit shelf life using deep learning techniques and method thereof.

The fruit shelf-life has been determined based on how much fruit is ripened and according to that classification has been done. As ripening is a biological change there must be a heat output or heat input that is an exothermic or endothermic reaction and thus there must be a relation with its surface temperature as temperature affects most of the biological processes in the development of fruits which in turn influences fruit-size, color, sugar content, acid content, starch content, smell, etc. Using thermal imaging without the destruction of fruit, it has been observed that as the fruit gets ripe, every day the temperature of the fruit gets increase. Thus according to this temperature difference of each day of fruit, we can identify the shelf life of fruit in accurate manner.

In an embodiment, a system for determining shelf life of a fruit is provided. The system includes a first receiver 202 for receiving an input from a user to take images of one or more fruits whose shelf life is to be ascertained; a thermal imaging device 204 configured to capture thermal images 206 of one or more fruits on receiving the input by the receiver 202, wherein said thermal imaging device 204 is provided with a motor configured to rotate 360 degrees to assist said thermal imaging device 204 capture images 206 of said one or more fruits from all directions, wherein said thermal imaging device 204 converts infra-red radiations released by the fruit and backgrounds and surrounds of said fruit into a visible thermal image 206; a first transmitter 208 operationally interconnected to a thermal imaging device 204 to transmit captured images by said thermal imaging device 204 to a server called as processing unit 210; a processing unit 210 configured to ascertain shelf life of said one or more fruits based on the received thermal images 206, wherein said processing unit 210 ascertains shelf life of one or more fruits; by: determining the maximum and minimum temperature value of the fruit along with the temperature distribution of entire fruit to differentiate whether scanned fruit is from cold storage or normal temperature automatically classifying acquired thermal images 206 of fruits into a plurality of classes means number of days the fruit will remain edible as particular fruit's shelf-life; wherein this is done by: comparing detailed temperature distribution with pre-defined threshold values also called as weights obtained by trained model by applying deep learning techniques on thermal dataset of the same standard fruit on the server 210 to determine the shelf life of the fruit; wherein thermal dataset comprises samples of several thermal images of fruits taken on every day after harvesting where fruit may be from cold storage or room temperature, these thermal images are then used to train the deep learning model; a display unit to display determined shelf life of the fruit.

In another embodiment, a process for determining shelf life of a fruit is provided. The process includes the steps of 102 receiving an input from a user to take images of one or more fruits whose shelf life is to be ascertained; 104 capturing thermal images of one or more fruits on receiving the input by the receiver, wherein said thermal imaging device is provided with a motor configured to rotate 360 degrees to assist said thermal imaging device capture images of said one or more fruits from all directions, wherein said thermal imaging device converts infra-red radiations released by the fruit and backgrounds and surrounds of said fruit into a visible thermal image; 106 transmitting captured images by said thermal imaging device to a server; 108 ascertaining shelf life of said one or more fruits based on the received images, wherein said ascertaining comprises: determining the maximum and minimum temperature value of the fruit along with the temperature distribution of entire fruit to differentiate whether scanned fruit is from cold storage or normal temperature automatically; classifying acquired thermal images of fruits into a plurality of classes means number of days the fruit will remain edible as particular fruit's shelf-life; comparing detailed temperature distribution with pre-defined threshold values also called as weights obtained by trained model by applying transfer learning a deep learning technique from the thermal dataset of the same standard fruit wherein thermal dataset is generated by capturing thermal images of particular fruit on every day after harvesting where fruit may be from cold storage or room temperature; this thermal image dataset is augmented to pretrained model of thermal images for transfer learning; pretrained weights are updated by newly trained model which are then used to classify newly sampled thermal image of a fruit for predicting that fruit's shelf life; 110 displaying determined shelf life of the fruit.

An object of the present invention is to provide a method and system for identifying fruit shelf life using deep learning techniques.

Another object of the present invention is to provide accuracy results related to fruit shelf life.

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### Technical Problem

The current methods of grading fruits based on maturity are mostly by visual inspection of physical characteristics like color, size, and shape and using traditional image processing methods for classification. Also, many analytical models have been technologically advanced for such a task using machine learning techniques where RGB imaging has the only basis which is not useful to find the internal damages of the fruits as well as not beneficial to determine shelf-life of fruits like Kiwi, Avocado, or Langra mango whose outer skin color remains same after it ripens. The existing system is slow based on Machine Learning. So, the major concern in the present system is finding the internal properties of the fruits nondestructively for identifying fruit's shelf-life accurately.

### Solution to Problem

In the solution, a method is disclosed for analyzing multiple significant maturity indices like color, ultra-structural changes in texture of the fruit to measure precisely the shelf life, optimum harvest time and develop a framework based on thermal imaging approach along with transfer learning a deep learning technique for feature extraction and classification of fruits. Low cost standalone farm management solutions can be built using this concept that will help multiple stakeholders in the fruit supply chain in improved quality control, grading and classification of fruits.

### Advantageous Effects of Invention

The major advantage of the design is it being a hand-held device with easy-to-use features and fast results. It is a lightweight with a robust framework for running the inference on mobile, as it imparts the results within a fraction of a second and also results in good generalization ability. Our design can be easily replicated and deployed over multiple devices and can help large group of workers to finish the task of grading in no time. The work process of the warehouses and fruit storage units gets automatized. It can give a clear picture to the managers about the condition of fruits at their dashboards. The design avoids a lot of manual and paper work, thus saving a lot of currency.

### BRIEF DESCRIPTION OF DRAWINGS

These and other features, aspects, and advantages of the present invention will become better understood when the following detailed description is read with reference to the accompanying drawings in which like characters represent like parts throughout the drawings, wherein:

FIG. 1 shows a flowchart for a process for determining shelf life of a fruit in accordance with an embodiment of the present invention;

FIG. 2 shows a block diagram for a system for determining shelf life of a fruit in accordance with an embodiment of the present invention by implementing the method illustrated in FIG. 1;

FIG. 3 shows proposed design of hardware system in accordance with an embodiment of the present invention;

FIG. 4 shows architecture of proposed system in accordance with an embodiment of the present invention;

FIG. 5 illustrates thermal images of fruit kept at different temperatures in accordance with an embodiment of the present invention;

FIG. 6 illustrates block diagram of working of thermal camera in accordance with an embodiment of the present invention;

FIG. 7 illustrates color bar of the fruit in accordance with an embodiment of the present invention;

FIG. 8 illustrates flow chart diagram of Deep learning technique with Transfer learning in accordance with an embodiment of the present invention;

FIG. 9 illustrates feature extraction fruit images in accordance with an embodiment of the present invention;

FIG. 10 illustrates exemplary output of shelf-life for particular fruit (RUL=Remaining Useful Life) in accordance with an embodiment of the present invention;

FIG. 11 illustrates RGB image, thermal image and infected damage images in accordance with an embodiment of the present invention; and

FIG. 12 illustrates example of Training and Test Accuracy as well as Training and Test Loss of fruit.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DESCRIPTION OF EMBODIMENTS

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present invention will be described below in detail with reference to the accompanying drawings.

The present invention generally relates to an image processing with the help of deep learning classification technique. In particularly, the present invention relates to a system for identifying fruit shelf life using deep learning techniques and method thereof.

FIG. 1 shows a flowchart for a process for determining shelf life of a fruit. The process includes the step 102 of receiving an input from a user to take images of one or more fruits whose shelf life is to be ascertained; Step 104 of capturing thermal images of one or more fruits on receiving the input by the receiver, wherein said thermal imaging device is provided with a motor to assist said thermal imaging device to rotate 360 degrees and capture images of said one or more fruits from all directions, wherein said thermal imaging device converts infra-red radiations released by the fruit and backgrounds and surrounds of said fruit into a visible thermal image; Step 106 of transmitting captured images by said thermal imaging device to a server; Step 108 of ascertaining shelf life of said one or more fruits based on the received images, said ascertaining process comprising determining the maximum and minimum temperature value of the fruit along with the temperature distribution of entire fruit to differentiate whether scanned fruit is from cold storage or normal temperature automatically; classifying acquired thermal images of fruits into a plurality of classes means number of days the fruit will remain edible as particular fruit's shelf-life; comparing detailed temperature distribution with pre-defined threshold values also called as weights obtained by trained model by applying transfer learning a deep learning technique from the thermal dataset of the same standard fruit wherein thermal dataset is generated by capturing thermal images of particular fruit on every day after harvesting where fruit may be from cold storage or room temperature; this thermal image dataset is augmented to pre-trained model of thermal images for transfer learning; pre-trained weights are updated by newly trained model which are then used to classify newly sampled thermal image of a fruit for predicting that fruit's shelf life; and Step 110 of displaying determined shelf life of the fruit.

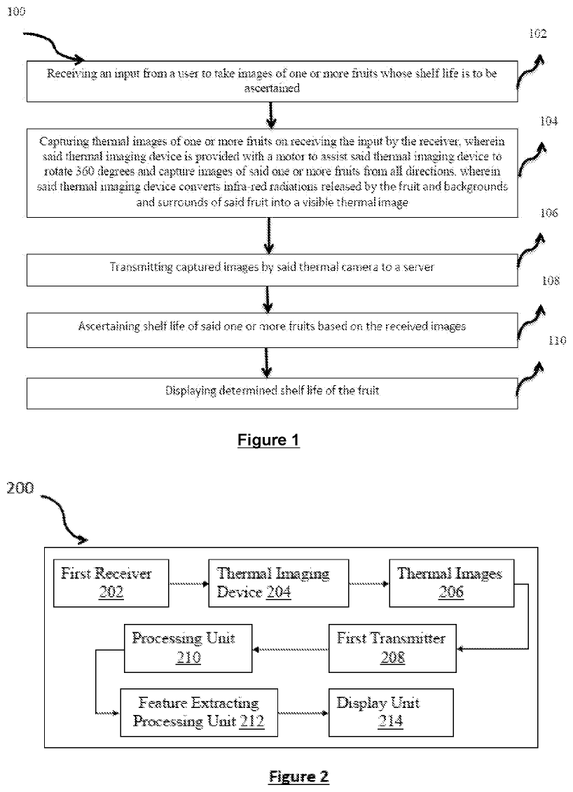

FIG. 2 shows a block diagram for a system for determining shelf life of a fruit in accordance with an embodiment of the present invention by implementing the method illustrated in FIG. 1. The system includes a first receiver 202 for receiving an input from a user to take images of one or more fruits whose shelf life is to be ascertained; a thermal imaging device 204 configured to capture thermal images 206 of one or more fruits on receiving the input by the receiver, wherein said thermal imaging device 204 is provided with a motor to assist said thermal imaging device 204 to rotate 360 degrees and capture images of said one or more fruits from all directions, wherein said thermal imaging device 204 converts infra-red radiations released by the fruit and backgrounds and surrounds of said fruit into a visible thermal image 206; a first transmitter 208 operationally interconnected to a thermal imaging device 204 to transmit captured images by said thermal imaging device 204 to a server called as processing unit 210; a processing unit 210 configured to ascertain shelf life of said one or more fruits based on the received thermal images 206, wherein said processing unit 210 ascertains shelf life of one or more fruits; by: determining the maximum and minimum temperature value of the fruit along with the temperature distribution of entire fruit to differentiate whether scanned fruit is from cold storage or normal temperature automatically classifying acquired thermal images 206 of fruits into a plurality of classes means number of days the fruit will remain edible as particular fruit's shelf-life; wherein this is done by: comparing detailed temperature distribution with pre-defined threshold values also called as weights obtained by trained model by applying deep learning techniques on thermal dataset of the same standard fruit on the server 210 to determine the shelf life of the fruit; wherein thermal dataset comprises samples of several thermal images of fruits taken on every day after harvesting where fruit may be from cold storage or room temperature, these thermal images are then used to train the deep learning model; a display unit 214 to display determined shelf life of the fruit.

In an embodiment, a feature extracting processing unit 212 which is method of convolution and pooling is configured to for extracting the features from the scanned thermal image, where training dataset of thermal images 206 has been trained with pre-trained model via transfer learning a deep learning technique. Using transfer learning, the trained network's (example: squeeze net or mobile net) weights have been used to classify the new thermal image data means thermal image of any new fruit to be tested for edibility in terms of number of days. The features of this new fruit's thermal image have been extracted from convolution and pooling layer of these pre-trained models.

FIG. 3 shows proposed design of hardware system in accordance with an embodiment of the present invention. The hardware system includes a thermal camera in machine application interface that captured different images of the fruits. A transmitter configured to send the captured images to a server. The server having a deep learning classification technique database that matches the captured images with pre stored images and shows the results related to the fruit shelf life.

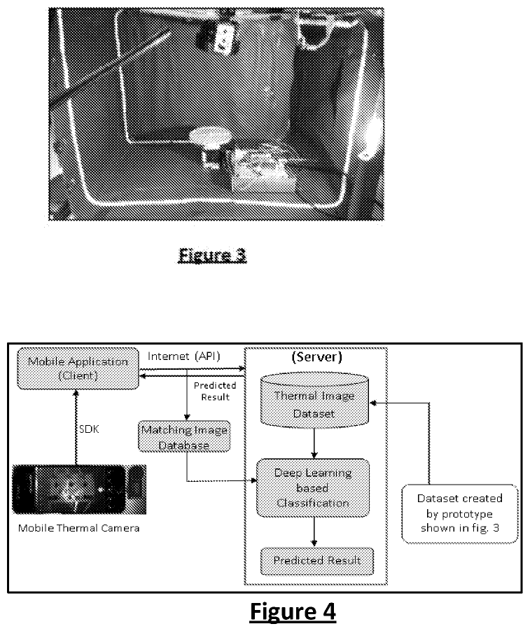

FIG. 4 shows architecture of proposed system in accordance with an embodiment of the present invention. The mobile phone client application takes the photograph using mobile thermal camera (Seek Thermal Compact and its SDK) and sends it to the server through Internet. The image received to the server will be matched with the trained set of deep learning classification and will send the result as fruit shelf to the client.

In an embodiment, design of hardware prototype will be used to capture Thermal images automatically from all the directions (0° to 360°). Design of software module will get the request from the client (i.e. from mobile application) and send the prediction results to client side mobile application. Mobile application will give prediction result of fruit shelf life.

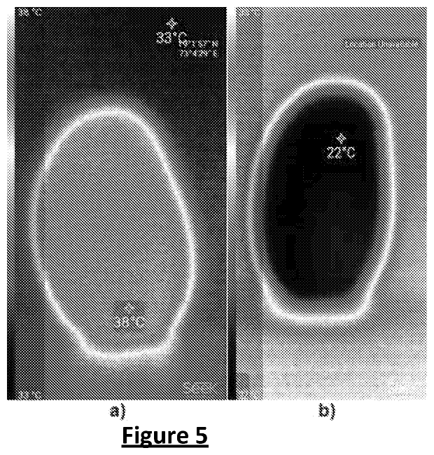

FIG. 5 illustrates thermal images of fruit kept at different temperatures in accordance with an embodiment of the present invention. FIG. 5a illustrates thermal image of fruit kept at room temperature (Ts=33 and TF=38) and FIG. 5b illustrates thermal image of fruit after kept in cold storage (Ts=33 and TF=22). The system is the combination of thermal imaging technology, deep learning technique with transfer learning algorithm and agricultural produce. As the fruit gets mature or riper; its temperature becomes high as compared to the immature or unripe fruit if the fruit is kept at room temperature, so day by day change in surface temperature becomes more. But if the fruit is in cold storage then temperature becomes less in mature or ripe fruit as compared to the immature or unripe fruit, so day by day change in surface temperature becomes less.

The equation to first find automatically whether the images are at room temperature or from cold storage by finding maximum (Tₘₐₓ) and minimum temperature (Tₘᵢₙ) of image. If surrounding temperature Tₛ is maximum and fruit temperature T_{F} is minimum then the temperature difference (ΔT) is ΔT≥1 for image from cold storage and if fruit temperature T_{F} is maximum and surrounding temperature Tₛ is minimum then the temperature difference (ΔT) is ΔT<1, for image at room temperature as described in Math1.

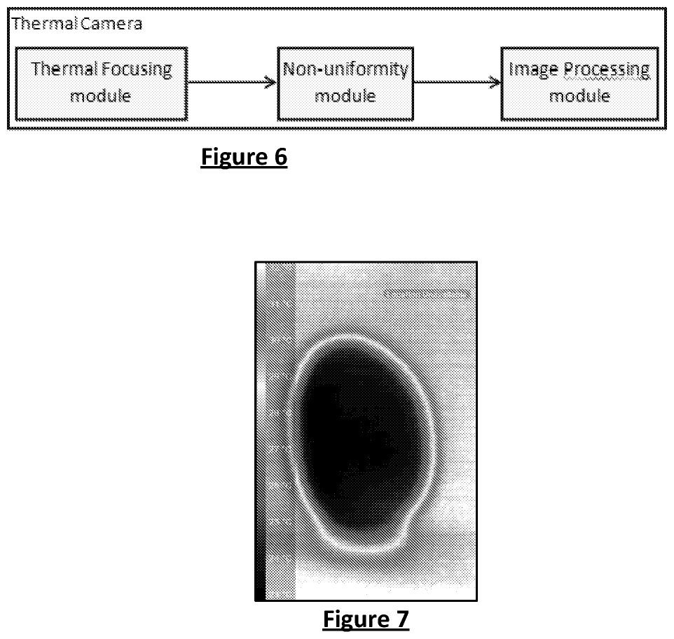

FIG. 6 illustrates block diagram of working of thermal camera in accordance with an embodiment of the present invention. In focusing module, analog signals received from the detectors converts into digital form. Non-uniformity module has sophisticated power sources for powering and biasing internal circuitry of focusing module and also special filters are there which enables noise immunity. In this module, correction of defective signal has been done using these filters. Image processing module manages control signals responsible for proper readout from non-uniformity module in matrix form. This module converts infra-red radiations released by any fruit and its backgrounds/surrounds into visible thermal image.

In an implementation, the advantage of thermal imaging is that it detects the thermal radiation and examines the fruits internally and externally without rupturing or dissecting it, without touching it, in a minimum span of time. It has the advantage of invariant to color change, lighting conditions, and also even works in the dark. Size of Thermal images is small compared to RGB images, so perform speedy processing.

In an implementation, the fruit shelf-life has been determined based on how much fruit is ripened and according to that classification has been done. As ripening is a biological change there must be a heat output or heat input that is an exothermic or endothermic reaction and thus there must be a relation with its surface temperature as temperature affects most of the biological processes in the development of fruits which in turn influences fruit-size, color, sugar content, acid content, starch content, smell, etc. Using thermal imaging without the destruction of fruit, it has been observed that as the fruit gets ripe, every day the temperature of the fruit gets increase. Thus according to this temperature difference of each day of fruit, we can identify the shelf life of fruit in accurate manner.

With the abovementioned details, the advantage of classifying the fruit shelf life using Thermal imaging with deep learning through transfer learning of pre-trained networks are speedy operations because of Thermal imaging as well as transfer learning (the size of Thermal images are small as compared to RGB imaging, so requires less time for processing).

FIG. 7 illustrates color bar of the fruit in accordance with an embodiment of the present invention. The color bar is present on the Thermal image which has been captured by thermal imaging device 204 which shows various temperature values has been detected from the particular fruit. The maximum and minimum temperature value of the fruit has been find out to differentiate whether scanned fruit is from cold storage or normal temperature; The thermal imaging device 204 converts infra-red radiations released by the fruit and backgrounds and surrounds of said fruit into a visible thermal image. Then this mapped thermal visible image is used for further processing like feature extraction, classification of fruits into number of classes means number of days the fruit will remain edible as particular fruit's shelf-life which has been decided for any type of fruit (example: for mango, classes are RUL_0 to RUL_18; for strawberry, classes are RUL_0 to RUL_3 etc.) and prediction of shelf life for particular fruit.

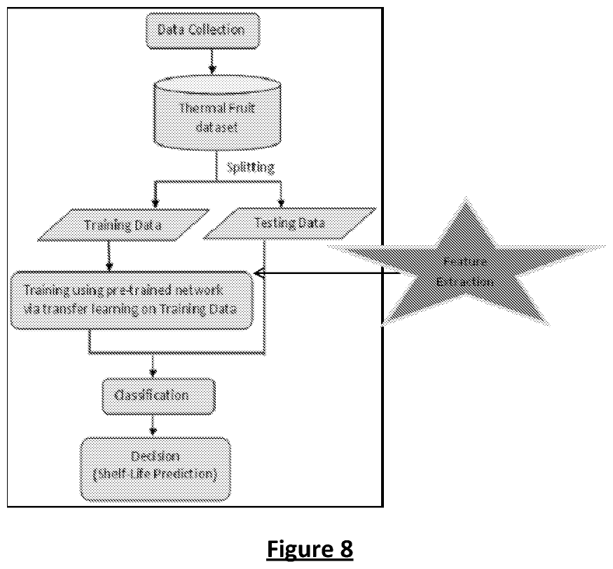

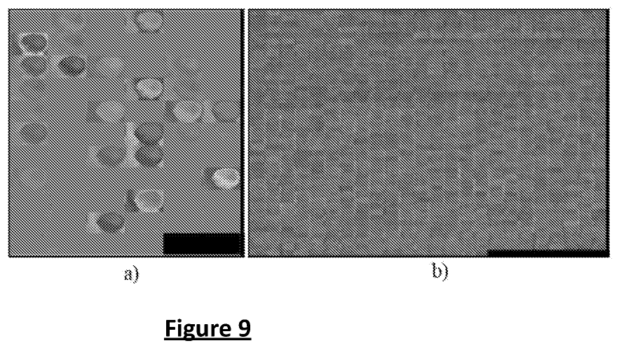

FIG. 8 illustrates flow chart diagram of Deep learning technique with Transfer learning in accordance with an embodiment of the present invention. The acquired Thermal images of fruits are classified into number of classes' according to particular fruit's shelf-life that is how many number of days are remaining to consume the fruit. The captured thermal images are resized according to the size of the pre-trained model used i.e. (227×227×3) or (224×224×3). This is useful for enhancement of accuracy and speediness in training and testing phase. Training dataset has been trained with pre-trained model via transfer learning a deep learning technique. Using transfer learning, the trained network's (example: squeeze net or mobile net) weights have been used to classify the new thermal image data. The features have been extracted from convolution and pooling layer of these pre-trained models for this new fruit's thermal image. Comparing with temperature threshold value also called as weights obtained by trained model by applying deep learning techniques, labels and its probabilities has been predicted for the new set of images.

In an implementation, deep Learning is the latest approach used in computer vision because of its capability of building precise models in a timesaving manner. It has a natural learning ability where there is no need of use any image processing feature extraction technique.

In an implementation, using deep learning techniques, we don't have to think of which image features like color, shape or texture are suitable for particular application. Also, to achieve better results, we required large amount of data but for transfer learning small amount of dataset is sufficient. Using transfer learning with pre-trained models, we can re-use model weights (as the starting point of training process) as feature extraction preprocessing, and integrated into entirely new models by re-training it for the new dataset (Thermal Dataset) which saves the time and improves the accuracy compared to image processing, machine leaning and deep learning without transfer learning. Transfer learning does not required as much labeled data.

FIG. 9 illustrates feature extraction fruit images in accordance with an embodiment of the present invention. FIG. 9a shows Low level 64 feature maps of fruit and figure b shows Abstract/High level feature map in accordance with an embodiment of the present invention. Initial convolution and pooling layer gives low level image features like color, edges, textures etc. and the last convolution and pooling layer gives abstract features. The feature map of one of the fruit is shown below where FIG. 9a is low level features like color, edges, textures etc. extracted from first convolution layer (number of features=64) and FIG. 9b is of features extracted from last convolution layer (number of features=512). The pre-trained models like squeeze net or mobile net have been used as these are the lightweight models which we can easily deploy for mobile application to get fast prediction results.

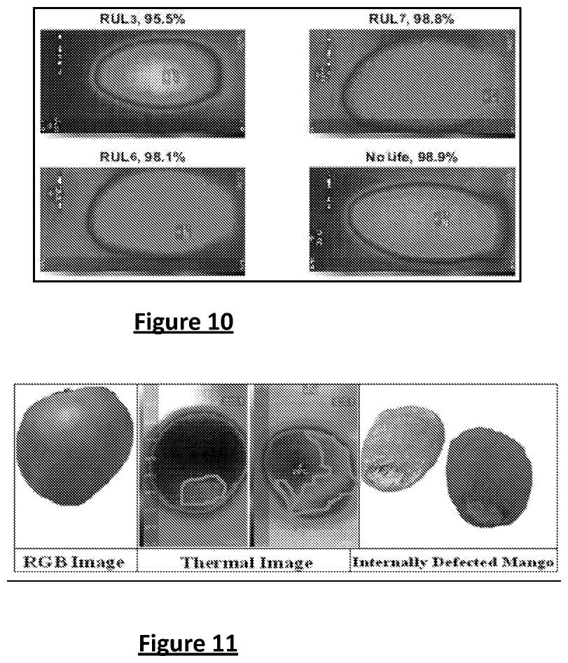

FIG. 10 illustrates exemplary output of shelf-life for particular fruit (RUL=Remaining Useful Life) in accordance with an embodiment of the present invention. RUL 7 means 7 days are remaining (or shelf life is 7) that means particularly how many number of days are remaining to consume a fruit is shown in FIG. 10.

In an exemplary implementation, if user wants to identify the shelf-life of mango fruit, first the user has to capture the Thermal image of it. Then captured Thermal input image sends to the server to find the particular match with the Thermal dataset of mangoes which is on the server. For example, in the case of mango, shelf-life is nearly 19 days. So accordingly user has 19 classes like Day 1 to Day 19 remaining useful life of mango to consume.

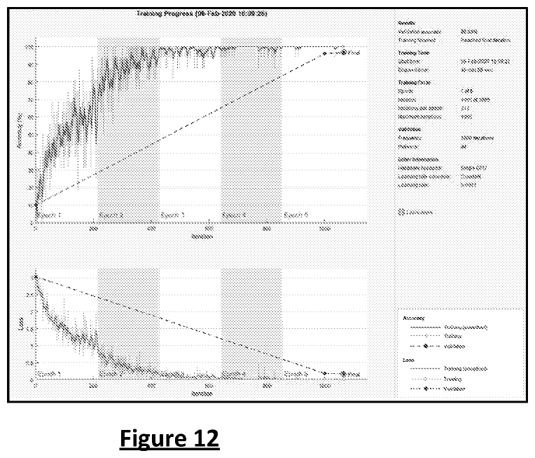

FIG. 11 illustrates RGB image, thermal image and infected damage images in accordance with an embodiment of the present invention. Camera capture photos or videos and then detects the fruits and first find whether fruit is defected or not internally or externally and then label it or evaluate it segregate it according to its life. So benefit is that at the primary level farmers or retailors can take the decision that whether to export the fruits or sold it at local market or upto what distance it is feasible to transport as the transportation with proper cold storage cost is more.

FIG. 12 illustrates example of Training and Test Accuracy as well as Training and Test Loss of fruit. As user observe in the above graph, both the training and test losses settle down rapidly for this dataset. The accuracy also increases in the range of 90's very fast where we can observe test accuracy as 96.93%. Finally, from the above result we can conclude that Thermal image is a good source of learning along with transfer learning through pre-trained models.

In an exemplary implementation, for experimental evaluation, mangoes fruits from different places may vary in their characteristics. The fruits have been inspected to check for damages. Then, they were washed and dried for 1 hour. The fruit has been labeled for identification purpose. The fruits have been kept in natural atmospheric conditions (temperature of 29° C.±2° C. and relative humidity of 72.4%±3%) for daily analysis. Daily at the same time weight of the fruit is noted using digital weighing scale and images are taken till the decay of fruit. It has been observed that if the size of fruit is small, medium and large, the weight loss becomes 25 to 40 gm, 28 to 45 gm and 35 to 60 gm respectively so because of which we can say loss in moisture content.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

### EXAMPLES

### Example 1

Example 1 describes output of shelf-life for particular fruit (RUL=Remaining Useful Life) in accordance with an embodiment of the present invention. FIG. 10 illustrates that RUL 7 means 7 days are remaining (or shelf life is 7) that means particularly how many numbers of days are remaining to consume a fruit.

In an exemplary implementation, if user wants to identify the shelf-life of mango fruit, first the user has to capture the Thermal image of it. Then captured Thermal input image sends to the server to find the particular match with the Thermal dataset of mangoes which is on the server. For example, in the case of mango, shelf-life is nearly 19 days. So accordingly user has 19 classes like Day 1 to Day 19 remaining useful life of mango to consume.

### Example 2

Example 2 describes Training and Test Accuracy as well as Training and Test Loss of fruit. FIG. 12 illustrates that as user observe in the graph, both the training and test losses settle down rapidly for this dataset. The accuracy also increases in the range of 90's very fast where we can observe test accuracy as 96.93%. Finally, from the above result we can conclude that Thermal image is a good source of learning along with transfer learning through pre-trained models.

In an exemplary implementation, for experimental evaluation, mangoes fruits from different places may vary in their characteristics. The fruits have been inspected to check for damages. Then, they were washed and dried for 1 hour. The fruit has been labeled for identification purpose. The fruits have been kept in natural atmospheric conditions (temperature of 29° C.±2° C. and relative humidity of 72.4%±3%) for daily analysis. Daily at the same time weight of the fruit is noted using digital weighing scale and images are taken till the decay of fruit. It has been observed that if the size of fruit is small, medium and large, the weight loss becomes 25 to 40 gm, 28 to 45 gm and 35 to 60 gm respectively so because of which we can say loss in moisture content.

$$Δ T = \frac{{T}_{s}}{{T}_{F}} \\ I = \{ \begin{matrix}Image from cold storage , Δ T \geq 1 \\ Image at room temperature , Δ T < 1\end{matrix} \qquad [ Math . 1 ]$$

### INDUSTRIAL APPLICABILITY

The consumption of fruits and their grading depends upon the quality of fruit and their shelf life. It becomes imperative to judge the quality of fruits for enhancing the net profits to be earned. The task of grading the fruit quality involves any of the three major methods namely: manual physical verification by experts or digital image processing-based identification or thermal imaging based on thermal imaging cameras. Most of the work in the fruit preservation units and warehouses is carried out manually in the absence of any automatic handheld tool for fast identification and grading. Here the role of our design comes to play. The design has been tried and tested on many fruits including the major important varieties of mango available in India. The design in this research shall enhance the overall functioning and speeding up of the work in fruit preservation units and warehouses and transportation units. These of deep learning-based techniques into the design has considerable improved the overall classification accuracy for the grading process. Therefore, our design can help managers and marketers save their precious time to accumulate data and manage their warehouses in no time. So, in all this helps agricultural industries to update their business processes.

### CITATION LIST

Citation List follows:

### PATENT LITERATURE

PTL 1 discloses mapping of temperature distribution with RGB grey values for enhancing the visibility of details in an image by using cyclic color space and the example is provided in the document by applying the same on thermal image where they have mapped the temperature values with RGB grey values; whereas in our invention, mapping of RGB colors to any color format is just a supporting step and not compulsory step to predict shelf life.

PTL1: Patent WO2007010531A2

### Non Patent Literature

NPL 1 refers to thermal imaging of fruits to examine the internal properties of fruits. The document discloses that thermal image sensor captures the thermal image. The image captured is then preprocessed and due to nonhomogeneous distribution of heat various regions of apples are shown clearly with different color. Thermal camera adds color to image for identification of irregular heat dispersion. Red color specifies the high temperature region; whereas in our invention, though our system makes use of thermal images as inputs they are not just used to find irregular heat dispersion looking at nonhomogeneous distribution of heat but such various images are taken as samples and their different features are extracted automatically with the help of convolution and pooling and training model is formed using transfer learning a deep learning technique; the trained model in terms of trained weights are used for predicting the shelf life means number of days the fruit will remain edible of any of the fruits scanned in real time; shelf life of any scanned fruit through thermal camera in terms of number of days is predicted precisely, which is not possible just through extracting heat distribution region scanned through thermal camera as mentioned in NPL1; thermal images are just acting as an input but they are processed differently than they are processed in NPL1 article; minimum and maximum temperature as well as entire distribution of temperature is taken into consideration and not the nonhomogeneous or irregular heat dispersion. This step is just for finding whether the images taken of fruit were from cold storage or normal temperature. It is a part of process; not only thermal images are captured but they are processed in real time and their shelf life is predicted using deep learning model via transfer learning; prediction of shelf life of fruits which is far different area than cited document NPL1 and finding whether fruit is defected or not is just a preliminary step before predicting shelf life; process for predicting fruit shelf life provides more accuracy and faster because of use of deep learning along with thermal imaging which will improve availability of fruits and prevent post-harvest loss in fruit industry as well as transportation problem.

NPL 2 relate to quality of food determination based on temperature distribution. The document discloses uneven distribution of temperature; the temperature distribution can be presented as the maximum, minimum, average temperature of the sample, as well as the central temperature of the apple slice where the uneven distribution of temperature is shown in different colors. Further analysis of the temperature distribution, demonstrates the minimum, maximum, center and average temperatures within the sample; whereas in our invention, the temperature is used to detect whether fruit to be tested for shelf life is from cold storage or from room temperature automatically which is totally different from temperature distribution discussed in document NPL 2 related to thermal image. It is one of the steps for determination of shelf life. Also, thermal images are taken and processed further for classification/prediction of shelf life without destruction of fruit which is major advantage of the system over document NPL 2 where fruit is destructed for observing thermal distribution.

NPL1: YOGESH ET AL.: ‘A comparative approach of segmentation methods using thermal images of apple’, IEEE, 2018

NPL2: JOARDDER ET AL.: ‘Effect of Temperature Distribution on Predicting Quality of Microwave Dehydrated Food’ JOURNAL OF MECHANICAL ENGINEERING AND SCIENCES. VOL. 5, 2013, pages 562-56

We claim:

1. A system for determining shelf life of a fruit, said system comprising:
a first receiver (202) for receiving an input from a user to take images of one or more fruits whose shelf life is to be ascertained;
a) a thermal imaging device (204) configured to capture thermal images (206) of one or more fruits on receiving the input by the receiver (202), wherein said thermal imaging device (204) is provided with a motor configured to rotate 360 degrees to assist said thermal imaging device (204) to capture thermal images (206) of said one or more fruits from all directions, wherein said thermal imaging device (204) converts infrared radiations released by the fruit and backgrounds and surrounds of said fruit into a visible thermal image (206);
b) a first transmitter (208) operationally interconnected to a thermal imaging device (204) to transmit captured images by said thermal imaging device (204) to a server called as processing unit (210);
c) a processing unit (210) configured to ascertain shelf life of said one or more fruits based on the received thermal images (206), wherein said processing unit (210) ascertains shelf life of one or more fruits; by:
determining the maximum and minimum temperature value of the fruit along with the temperature distribution of entire fruit to differentiate whether scanned fruit is from cold storage or normal temperature automatically;
classifying acquired thermal images (206) of fruits into a plurality of classes means number of days the fruit will remain edible as particular fruit's shelf-life;
wherein this is done by:
comparing detailed temperature distribution with pre-defined threshold values also called as weights obtained by trained model by applying deep learning techniques on thermal dataset of the same standard fruit on the server (210) to determine the shelf life of the fruit; wherein thermal dataset comprises samples of several thermal images of fruits taken on every day after harvesting where the fruit may be from cold storage or room temperature, these thermal mages are then used to train the deep learning model; a display unit (214) to display determined shelf life of the fruit;

2. The system as claimed in claim 1, wherein a feature extracting processing unit (212) which is method of convolution and pooling is configured to extract the features from the scanned thermal image, wherein a training dataset of thermal images (206) is trained with a pre-trained model via transfer learning, a deep learning technique, and wherein using transfer learning, the trained network's (example: squeeze net or mobile net) weights are used to classify the new thermal image data means thermal image of any new fruit to be tested for edibility in terms of number of days, and wherein features of this new fruit's thermal image have been extracted from convolution and pooling layer of these pre-trained models.

3. A process for determining shelf life of a fruit, said process comprising:
(102) receiving an input from a user to take images of one or more fruits whose shelf life is to be ascertained;
(104) capturing thermal images of one or more fruits on receiving the input by the receiver (202), wherein said thermal imaging device (204) is provided a motor configured to rotate 360 degrees to assist said thermal imaging device capture images of said one or more fruits from all directions, wherein said thermal imaging device (204) converts infra-red radiations released by the fruit and backgrounds and surrounds of said fruit into a visible thermal image;
(106) transmitting captured images by said thermal imaging device (204) to a server (210);
(108) ascertaining shelf life of said one or more fruits based on the received images, wherein said ascertaining comprises:
determining the maximum and minimum temperature value of the fruit along with the temperature distribution of entire fruit to differentiate whether scanned fruit is from cold storage or normal temperature automatically;
classifying acquired thermal images of fruits into a plurality of classes means number of days the fruit will remain edible as particular fruit's shelf-life;
comparing detailed temperature distribution with pre-defined threshold values also called as weights obtained by trained model; by applying transfer learning a deep learning technique; on the thermal dataset of the same standard fruit; wherein thermal dataset is generated by capturing thermal images of particular fruit on every day after harvesting where the fruit may be from cold storage or room temperature; this thermal image dataset is augmented to pretrained model of thermal images for transfer learning; pretrained weights are updated by newly trained model which are then used to classify newly sampled thermal image of a fruit for predicting that fruit's shelf life.
(110) displaying determined shelf life of the fruit.
